(19) United States
(12) Patent Application Publication
(10) Pub. No.: US 2005/0271228 A1
(43) Pub. Date: Dec. 8, 2005

(54) Handheld device with hearing aid function

(75) Inventors: Cheng-Shing Lai, Taipei Hsien (TW); Rong-Lin Qin, Nanking (CN); Fitch Fan, Nanking (CN)

Correspondence Address: BACON & THOMAS, PLLC, 625 SLATERS LANE, FOURTH FLOOR, ALEXANDRIA, VA, 22314, US

(73) Assignee: Inventec Appliances Corporation, Taipei Hsien (TW)

(21) Appl. No.: 10/859,193

(22) Filed: Jun. 3, 2004

(51) Int. Cl. A61M 21/00; H04R 25/00

(52) U.S. Cl. 381/312; 381/316; 381/322

(57) ABSTRACT

The present invention discloses a handheld device using a sound receiver, such as a mobile phone to receive a sound in the surrounding within an appropriate range, and after the sound volume is amplified appropriately, a source playback device such as an earphone is used to play the amplified sound, so that a person having a hearing problem can just carry a handheld device to have the basic functions of the handheld device such as sending and receiving a short message and also use the sound amplification function thereof to supplement the deficiency of the hearing ability when talking to or communicating with others.

### FIELD OF THE INVENTION

The present invention relates to handheld devices, more particularly to a handheld device capable of receiving a sound in the surrounding, appropriately amplifying the sound volume and playing the amplified sound out, so that a person having a hearing problem can just carry a handheld device to have the basic functions of the handheld device and also use the sound amplification function thereof to supplement the deficiency of the hearing ability when talking to or communicating with others.

### BACKGROUND OF THE INVENTION

When an object vibrates, it will drive the surrounding air molecules to vibrate accordingly and will also create waves with different densities. This kind of air wave is called sound wave, and the vibrating object is referred as the sound source. When a sound wave is transmitted to our ears, our hearing system will receive and convert the energy transmitted by the sound wave, and then our nervous system will transmit the energy to our cerebrum to sense the sound after the energy is identified by the cerebrum. And that is the sound we hear in our daily life.

The sound wave enters our outer ear, middle ear, and inner ear one by one, and then enters our brainstem and cerebrum. If there is a problem with any step along the whole process, we probably will lose our hearing. More specifically, the main function of our outer ears and inner ears is to collect and transmit sound waves. As to the cochlea of the inner ear, its main function is to convert the energy of the received sound wave into a nerve signal, and the nerve signal is transmitted from the hearing nerve to the central nervous system. If there is a hearing problem caused by the hindrance at the outer ear or the middle ear, it is called the conductive hearing loss. If the hearing problem is caused by the hindrance at the cochlea of the inner ear or the injury of the hearing nerve, it is called the sensorineural hearing loss. If the hearing problem is caused by both of the above, it is called mixed hearing loss. Generally, the conductive hearing loss can be cured by medicines or surgical operations, and the sensorineural hearing loss can only be overcome by wearing a hearing aid.

What is a hearing aid? For simplicity, a hearing aid is a sound amplifier. Let us simulate the following common sound amplifier that comes with a microphone, a main system for amplifying the volume of a sound, and a speaker. These three components are assembled and reduced to the size in order to fit our ears. That is a hearing aid. The idea of the hearing aid is to amplify the sound, so that the originally unheard sound can be heard. In fact, this idea is commonly used in our daily life. For example, we usually raise our voice when communicating with old folks having a poor hearing ability or speak close to their ears to enhance the sound effect to their ears. With the help of hearing aids to amplify the sound, we need not to raise our voice anymore.

Since the development of mobile phones is very fast, and almost everyone has a mobile phone in these days. Therefore, a person having a hearing problem still can use a mobile phone to talk directly with others by wearing a hearing aid. Even though such person has not worn a hearing aid, that person still can communicate with others in a text form by adopting the short message service function of the mobile phone.

Since mobile phones are very common and popular in our daily life, therefore if a person having a hearing problem can use a mobile phone with sound amplification function as a hearing aid to hear the change of sound in the surrounding. Further, the person having a hearing problem can hear the caller's voice clearly when receiving a call by the mobile phone. Such invention is definitely a great contribution to the people who have a hearing problem.

### SUMMARY OF THE INVENTION

The primary objective of the present invention is to use a sound receiver installed in a handheld device (such as a mobile phone) to receive a sound in the surrounding within an appropriate range, and after the sound volume (amplitude of the sound) is amplified appropriately, a source playback device (such as an earphone) is used to play the amplified sound, so that a person having a hearing problem can just carry a handheld device to have the basic functions of the handheld device (such as sending and receiving a short message) and also use the sound amplification function of the handheld device to supplement the deficiency of the hearing ability when talking to or communicating with others.

Another objective of the present invention is to install a save/reply control device into the electronic handheld device, such that the sound received and amplified by the handheld device can be recorded and saved into a data storage medium for the person having the hearing problem to listen to the recording anytime.

The foregoing ingenious idea simulates a handheld device as a hearing aid to assist people who have a hearing problem.

To make it easier for our examiner to understand the objective of the invention, its shape, structure, innovative features, and performance, we use a preferred embodiment together with the attached drawings for the detailed description of the invention.

### BRIEF DESCRIPTION OF THE DRAWINGS

FIG. 1 is a structural block diagram of the present invention.

FIG. 2 is a schematic view of a cable earphone according to the present invention.

FIG. 3 is a schematic view of a wireless earphone according to the present invention.

### DETAILED DESCRIPTION OF THE PREFERRED EMBODIMENTS

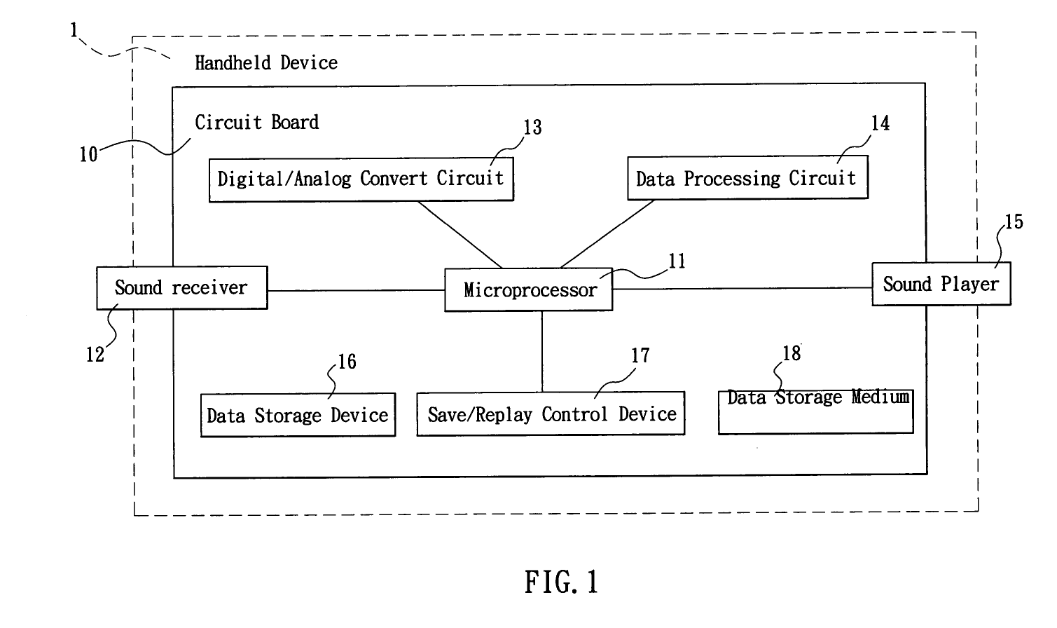

Please refer to FIG. 1 for the electronic handheld device with a hearing aid function according to the present invention. The electronic handheld device 1 comprises a circuit board 10 therein and the circuit board 10 has a microprocessor 11, a sound receiver 12, an analogy/digital convert unit 13, a data processing unit 14, and a sound player 15; wherein the microprocessor 11 converts a primitive analog signal (such as the voice in a telephone conversation) received by the sound receiver 12 into a primitive digital signal by the analog/digital convert circuit 13. The volume (also known as amplitude) of the original digital signal is amplified by the audio data processing unit 14 appropriately, and the noises of the original signal are filtered to produce an amplified digital signal. The amplified digital signal is converted into an amplified analog signal by the analog/digital convert circuit 13 and then played by the sound player 15.

With the foregoing ingenious design, a patient having a hearing problem can use or operate a handheld device 1 to save the cost of buying an additional hearing aid. The person having a hearing problem can use the handheld device 1 to talk with others at regular time, or appropriately amplify the other's voice to assist and enhance the hearing ability. Therefore, the present invention is definitely an economic and perfect idea to benefit the people who have a hearing problem.

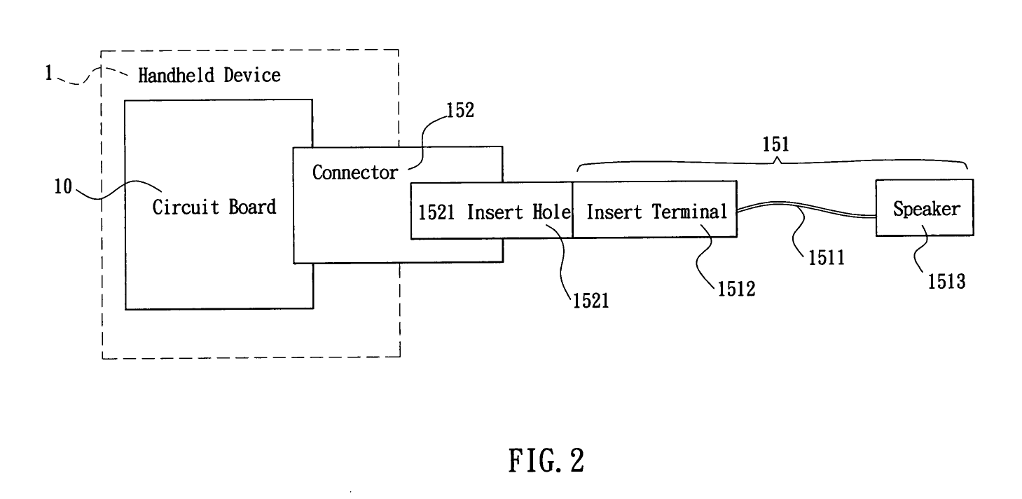

Please refer to FIGS. 1 and 2 for the sound player 15 of the present invention, which comprises a cable earphone 151 and a connector 152 (as shown in FIG. 2), wherein the connector 152 connected to the circuit board 10 has an insert hole 1521, and the cable earphone 151 has a signal line 1511, and one end of the signal line 1511 is an insert terminal 1512, and the other end is connected to at least one speaker 1513. When the insert terminal 1512 is inserted into the insert hole 1521, the amplified analog signal passes through the connector 152, the insert terminal 1512, and the signal line 1511 in order, and finally played from the speaker 1513.

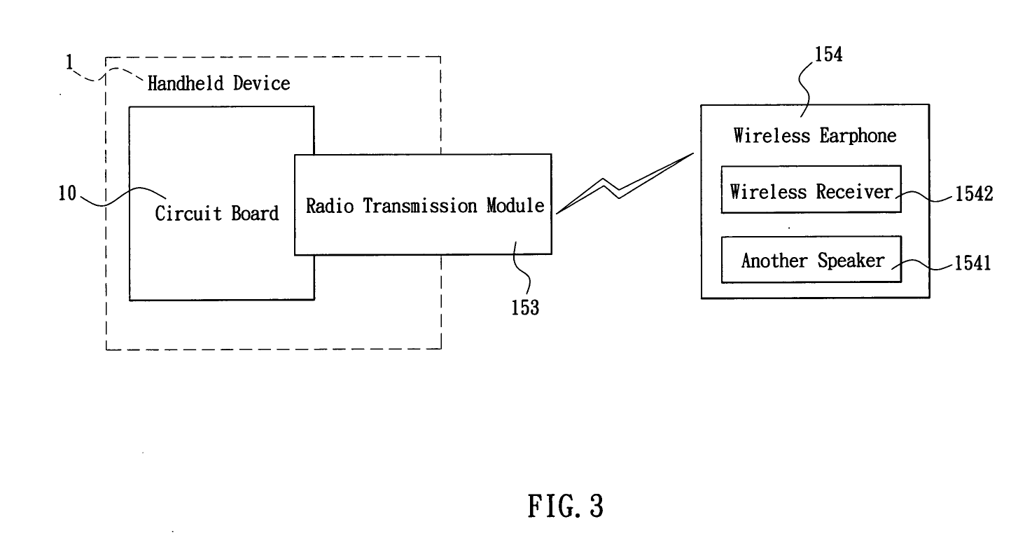

Please refer to FIGS. 1 to 3 for another preferred embodiment of the present invention. Since the signal line 1511 is limited to a specific length and a user having a hearing problem cannot place the handheld device 1 at a position far away from the user while the user is wearing a cable earphone 21, therefore the sound player 15 further comprises a radio transmission module 153 and a wireless earphone 154 (as shown in FIG. 3); wherein the wireless earphone 154 at least has another speaker 1541 therein, and the radio transmission module 153 works together with a wireless receiver 1542, so that after the amplified digital signal is converted back to the amplified analog signal by the analog/digital convert circuit 13, the amplified analog signal is transmitted to the wireless receiver 1542 through the radio transmission module 153 and played from another speaker 1541. Therefore, a person who has a hearing problem just needs to wear a wireless earphone 154 and put the handheld device 1 in an appropriate direction or at a farther position for receiving the sound without being limited by the length of the signal line 1511.

Since some people have a hearing problem on both ears, but the hearing ability is different on both ears, therefore a channel control device 16 (such as a digital signal processor, DSP) is installed on the circuit board 10 for eliminating noises, balancing frequencies, and adjusting the volume of sound on the left and right channels, so that the person having a hearing problem can use the channel control device 16 to adjust the sound volume of the left and right channels of the cable earphone 151 or the wireless earphone 154.

Further, a save/replay control device 17 and a data storage medium 18 are installed on the circuit board 10; wherein the save/replay control device 17 can save the amplified digital signal into the data storage medium 18. If the user wants to replay the amplified digital signal, the user can use the save/replay control device 17 to send the amplified digital signal to the analog/digital convert circuit 13 to reverse the amplified digital signal into the amplified analog signal and play the amplified analog signal from the sound player 15.

Please refer to FIGS. 1 to 3 for the present invention. The electronic handheld device 1 could be a mobile phone, a personal digital assistant (PDA) or a smart phone integrating the functions of a handset and a PDA; the data storage medium 18 could be a memory module or a memory card; and the wireless transmission module 153 could be an infrared module or a Bluetooth wireless module.

While the invention has been described by means of specific embodiments, numerous modifications and variations could be made thereto by those skilled in the art without departing from the scope and spirit of the invention set forth in the claims.

1. A handheld device with hearing aid function having a circuit board disposed therein and a microprocessor disposed on said circuit board, and said microprocessor being coupled to the following components for a signal connection:
a sound receiver, for receiving an analog signal transmitted from a surrounding environment within a finite range;
an analog/digital convert circuit, for converting a received audio signal selectively into an analog signal and a digital signal;
a data processing circuit, for appropriately amplifying the volume of said digital signal and filtering noises; and
a sound player, for playing an analog signal with an amplified volume.

2. The handheld device of claim 1, wherein said sound player comprises:
a cable earphone, having a signal line, and a connecting terminal disposed at an end of said signal line and at least a speaker disposed at the other end of said signal line; and
a connector, coupled with a circuit board, and having an insert hole thereon, so that said connecting terminal being inserted into said insert hole.

3. The handheld device of claim 1, wherein said sound player comprises:
a wireless ear phone, having a radio receiver and at least one other speaker thereon; and
a radio transmission module, for sending an analog signal after its sound volume is amplified to said radio receiver, so that said other speaker plays said analog signal.

4. The handheld device of claim 2, wherein said circuit board comprises a channel control device for adjusting the sound volume of said left channel and said right channel of said speaker.

5. The handheld device of claim 3, wherein said circuit board comprises a channel control device for adjusting the sound volume of said left and right channels of said other speaker.

6. The handheld device of claim 1, wherein said circuit board comprises a save/replay control device and a data storage medium for saving said digital signal after being amplified into said data storage medium.

7. The handheld device of claim 1, wherein said handheld device is a mobile phone.

8. The handheld device of claim 1, wherein said handheld device is a personal digital assistant.
